(19) United States
(12) Patent Application Publication
(10) Pub. No.: US 2016/0113801 A1
(43) Pub. Date: Apr. 28, 2016

(54) DYNAMIC HIP BRACE, HIP BRACE KIT AND METHOD OF USE

(71) Applicant: ANDREA WEISSLEDER, Sarasota, FL (US)

(72) Inventor: ANDREA WEISSLEDER, Sarasota, FL (US)

(21) Appl. No.: 14/524,180

(22) Filed: Oct. 27, 2014

(51) Int. Cl. A61F 5/01

(52) U.S. Cl. CPC A61F 5/0104

(57) ABSTRACT

Hip brace for motion control and support of the hip joint, and/or to the surrounding sacroiliac joint and/or lumbo-sacral joints concomitantly, built of configured elasticized neoprene with tensile strength, and with closures built of Velcro whose combined forces impart dynamic action and protection from hip dislocation and subluxation; range of motion control, joint protection and support, pain management, proprioceptive cueing, alignment support, resistive and assistive exercise options for people with disorders of the hip joint and as well for those with concomitant sacroiliac and lumbo-sacral instability. Medical conditions addressed include but not limited to hip pain, status post surgical hip, hip dislocation/subluxation, arthritis, sprains, strains, bursitis, tendonitis, joint hypermobility, ligamentous tears and sacroiliac and/or lumbo-sacral pain and instability, radiculitis as well as for athletes in need of hip joint and/or the sacroiliac, lumbo-sacral joint protection and support on and off the field and for everyday activities of daily living (ADL) and exercise.

### CROSS-REFERENCE TO RELATED APPLICATIONS

The present patent application claims the priority benefit of U.S. provisional patent application No. 61/895,588 filed on Oct. 25, 2013, the disclosure of which is incorporated by reference herein.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

When the hip joint is painful, vulnerable, weak, or subject to dislocation, the bracing devices most commonly used to this date are constructed of rigid or semi-rigid materials: metal uprights and leather over metal bands or plastic materials; often an exterior mechanical joint that allows only moderate free motion from a neutral position. These braces do not allow for any rotational hip range of motion. They are uncomfortable and difficult to manage; often subjecting a patient to blisters and sores from skin pressure from the firm brace materials pressing onto the skin. Many patients end up not using them. Sitting poses comfort issues with the rigid components, where as this device holds the L/S, S/l and hips a comfortably supported in sitting position.

Other designs have used elasticized materials and Velcro closures known commonly 1,2, albeit not in this proposed design and pattern (FIG. 1,2,3,4) for the body of the brace and application using Velcro and optional ancillary neoprene with Velcro straps 1,2,10, FIG. 7,9 for positioning and support in these recommended configurations (FIG. 1-22). There is a need for a comfortable, effective brace of soft materials; whereas the brace is constructed of soft, yet firm neoprene providing tensile strength; cut into a specific pattern (FIG. 1,2,3,4,5) wrapped around the lower torso at the waist 3 with the neoprene or like elasticized fabric with tensile strength bands coming together in a specific direction 14 and positioned and overlapping to fit snuggly (FIG. 5) and then secured with Velcro hook and loop straps 2,10,15, or like material pulling dynamically 14 in specific directions as per needed application. The thigh band 4 of same construction is wrapped around the thigh of the effected leg 11 in a specific configuration for each individual's fit and for the individual's deficit and purpose, be it general pain management, ambulation support, exercise (FIG. 15,16,17) containment of hot, cold or compression packs 19 (FIG. 9,22,23), to address specific muscles or muscle groups, to adjust to the strength or lack thereof, the neoprene Velcro ended straps 2,18 can be applied 17 strategically to the body of the brace. The straps are adhered in a particular configuration as per treatment rationale. (FIG. 1-22) The properties of elasticity and tensile strength of the neoprene of the brace body itself (FIG. 1,2,3) the firm, but adjustable Velcro closures (FIG. 8,9) and ability to stretch in the direction of pull 14 of the additional neoprene Velcro ended straps (FIG. 7,8,14) adjusted to the individual's contour and adhered to the brace body 14,15,16 determines the fit of the brace and thereby the security of the hip joint, as well as the stability of the sacroiliac and lumbo-sacral joints as the hip joint function and position is influenced by the security of the other surrounding joints—sacroiliac and lumbo-sacral joints. (FIG. 18,19,20,21) 32,30,33. These joints are also secured with this brace using additional neoprene strap or straps of any needed length or width as cut to size is easy, with Velcro closures with an alligator configuration or sewn on 18 placed around the ends of a neoprene strap and then strategically onto the body of the brace 5.

### FIELD OF INVENTION

Dynamic hip brace, hip brace kit to provide support and stability to a person's hip, lumbo-sacral spine, and sacroiliac.

### PRIOR ART

| Cite No | Pat. No. | Issue Date | Names of patentees or Applicant |
|---|---|---|---|
| 1 | 4,901,701 | 1990 Feb. 20 | Meyer |
| 2 | 4,905,678 | 1990 Mar. 08 | Cumins and Randolph |
| 3 | 4,926,845 | 1990 May 22 | Harris |
| 4 | 4,953,856 | 1990 Sep. 04 | Fox, III, Charles E. |
| 5 | 5,267,928 | 1993 Dec. 07 | Barile and Sawa |
| 6 | 5,419,755 | 1995 May 30 | Fukomoto et al |
| 7 | 5,840,050 | 1998 Nov. 24 | Lerman |
| 8 | 6,210,353 | 2001 Apr. 03 | Barnes |

| | | | | Name of |
|---|---|---|---|---|
| Cite No | Application Number | Kind Code | Publication Date | Applicant |
| 1 | 20020032397 | A1 | 2002 Mar. 14 | Coligado |
| 2 | 20060015049 | A1 | 2006 Jan. 06 | Suarez |
| 3 | 20060074365 | A1 | 2006 Apr. 06 | Brown |
| 4 | 20040116260 | A1 | 2004 Jun. 17 | Drennan |
| 5 | 20070027419 | A1 | 2007 Feb. 07 | Drennan |
| 6 | 20080072629 | A1 | 2008 Mar. 27 | Gehring |

Drennan US 2007/0027419 and its predecessor US20040116260 use neoprene fabric and Velcro as in this described embodiment but Drennan adds narrow round cables and a system of longitudinal loops, zipped on mesh fabric onto a soft device embodiment that is lined with soft material. There is an elaborate system of loops and cables. The individual cables are intended to thread through selected loops and drawn through the multiple longitudinal rows of loops embedded into a neoprene waistband, thigh cuff (s) and the body of the brace, then attaching with metal fasteners. These multiple elastic cables are threaded through the loops and are contained in a desired longitudinal path to effect a particular chosen pulling force to position the parts of the hip joint to help prevent dislocation. A lateral mesh strip joins the waist and thigh components together with zippers. There is a layer of fabric to go against the skin and the neoprene on the outer side. The 2007 entry adds shoulder straps to help prevent migration of the thigh cuff. Cables are used with or without a covering of neoprene or other such fabrics.

This described hip brace, application Ser. No. 12/454,375 is a marked improvement of a means to obtain the outcome of positioning the femoral head into the pelvic acetabulum to prevent dislocation but with different materials, method and organization of components, and it has other pertinent beneficial uses. Drennan's brace uses narrow cables which thread through narrow loops. The cables can course over bony prominences, which is threatening to the skin tissue below. The course of longitudinal cables must be moved if one or more of these cables pass over the buttocks, as sitting would be very uncomfortable. There was no discussion on how the patient would put it on. There is no discussion how the patient can adjust this device independently, which is important as the majority population with hip problems are elderly. They have very fragile skin and the tendency to sit. The unbroken loop neoprene (UBL) has a soft texture on both sides equally and no liner is required. Comfort is extremely important for compliance and for the health of the skin below. The neoprene will not bunch up or move around like liners as the fabric is anchored with double sided Velcro patches. Cables pose a liability to the skin below, not evident when using wide bands of neoprene and Velcro. The hip brace of application Ser. No. 12/454,375 purposely uses flat double sided bands of neoprene body, bands and straps with alligator configuration of Velcro which wraps at the end of a strap or band. Velcro can attach anywhere on the embodiment, can be removed and moved easily, targeting alignment of the hip joint, countering the forces causing dislocation and support of the lumbo-sacral spine and sacroiliac; specific muscle groups; assistive and resistive exercise, redistribution of the pressure on the femoral head so that arthritic people can bear weight without pain. The body of the brace with its contiguous waist and thigh bands, wrap around as horizontal bands and is a single entity of double-sided unbroken loop (UBL) neoprene, with additional straps of Velcro ended neoprene straps, permit more finite adjustments as these bands and straps attach anywhere on the body of the brace or other neoprene straps and also uses the pressure of the elasticized neoprene of the brace body itself to provide general support and delivers cues to the patient's body for support and proprioception. The double-sided neoprene straps with alligator closures allow for attachment to other straps as well. The brace also has velcro patches which are secured under straps or bands to prevent any motion. They are called anti-slip patches. Included are small circular Velcro pieces which adhere to the brace to designate the location where a patient can places straps day to day. The horizontal band and portion of the body of the brace with applied angular straps, supports the lumbo-sacral spine and sacroiliac as well—directly or concomitantly as the lower quarter of the torso should be addressed when rehabilitating the hip. There is no such option with the Dennan brace.

Drennan's elaborate use of multiple cables and the loops that they course through, define the forces to create desired alignment and with the use of shoulder straps and zippers to link the lower torso to the thigh band, and an under surface liner create a cumbersome combination of components.

With this described brace of application Ser. No. 12/454,375, there is no separation of the waist band and the thigh band. Therefore no zippers or mesh are required. Broad Velcro ended neoprene straps make it easy for the patient to don the brace independently. There is a simple method for making adjustments; remove and move straps so that the patient can make changes independently. There are an infinite amount of adjustments possible as the unbroken loop neoprene can effect more finite adjustments as the straps and bands attach anywhere on the body of the brace. The horizontal and/or angular bands and straps in addition to the body of the brace, offer support for the lumbo-sacral spine and sacroiliac.

Dennan does not add any for insertion firm components.

Barile's exercise garment uses some of the same materials of this described brace, albeit in a different way. He makes good use of the softer materials like neoprene and Velcro but in a completely different configuration with a focus on general rehabilitation, not for the immediate post surgical patient, IE.; dislocaters. Nevertheless, there is an elaborate use of neoprene straps and Velcro attachments. However, the attachments are sewn on various places onto a very short pant as opposed to straps fastening with Velcro loop and hook to the body of the brace. Velcro straps must target these sewn on locations only, which may miss the specific combination to get the precise desired position, IE; to prevent hip dislocation. Use depends upon the Velcro attaching to a flimsy pant. The pant itself is way too short and stretchy which will move easily and compromise the mechanics. Thus, there is no use of neoprene as a contributor of stability. The neoprene body of the application Ser. No. 12/454,375 brace hereto described is built as part of the equation for combining forces for a specific outcome. With availability of a longer lever arm, only if the pant or brace is longer and secured with something more than a drawstring, with the pulling force extend over more of the body of the brace can it can target distant reaches and attachments which defines the desired outcome of forces. It is it mechanically more advantageous to facilitate motion/motion control to have a firm embodiment that will help to contain the specific desired forces of the pull of Velcro. There is no use of a waistband to be part of the combination of forces. A drawstring is inadequate. The lumbo-sacral and sacroiliac require strong attachment anchors. The pants and thigh openings are cinched with a drawstring, thus eliminating any waistband security and or support for the pelvis; IE: sacroiliac and lumbo-sacral spine. It does not address the problem of hip dislocation or hip care specifically. The forces available to use Barile's device for exercise is dependent upon where the receptive Velcro is sewn onto a flimsy pant fastened with a drawstring, making fine-tuning difficult. There is no focus on preventing dislocation of the hip joint, sacroiliac or lumbo-sacral spine. This is a general exercise garment for anyone, but not specifically for the hip patient. There is no capcity for adding firm components.

Gehrig, George: US-2008/0072629A1 teaches a description of the neoprene fabric itself and how it is created with specifics on the variable uses of the width and height of threads of the fabric. Neoprene is the material of choice for this described hip brace. However, creating braces for specific purposes was not the intention of this invention description, rather a description of the many helpful ways this is fabric used. There was no mention of a hip brace application precisely. There was an acknowledgment of one of the uses of neoprene is for orthopedic devices, but no specific mention of hip bracing.

Fox, III, Charles E. describes an exercising garment that is made for muscular strengthening with and upper portion for the arms and upper torso and a lower torso portion and for the lower extremities. There is an elaborate series of straps and loops where weights can be attached. The rationale and order of these components serve for strengthening in general and also to improve form. The garment with its weights can be used during ambulatory exercise. There is no attempt to construct a hip brace in as in this description. There is no specified lumbo-sacral or sacroiliac support. There is no discussion of any garment or treatment to prevent hip dislocation.

### BRIEF SUMMARY

Dynamic Hip Bracing for the hip joint using elastisized soft materials and/or with added firm components that are comfortable and effective in providing dislocation protection, motion control and support, pain management; and/or for the sacroiliac and lumbo-sacral spine concomitantly with components for adjusting position precisely and provide for resistive/assertive exercise by virtue of the. The described dynamic hip brace and hip brace kit provides an effective means of preventing hip dislocation or subluxation of the hip joint by supporting, and controlling of provocative motions that often result in dislocation or subluxation; to afford a capability for exercise, both resisted and assisted to strengthen weakened muscles, especially those that can help to prevent dislocation; provides for possible normal gait while wearing; can provide for support to the lumbo-sacral spine and sacroiliac concomitantly and supporting the weight of the leg in acute lumbar radiculopathy. This described hip brace uses soft materials that are comfortable yet has the tensile strength for effectively protecting hip dislocation, motion control and support, pain management; and/or for the sacroiliac and lumbo-sacral spine concomitantly can include added components for precisely adjusting hip position, support, and resistance or assistance for exercise or motion control.

### DRAWINGS AND FIGURES

For purposes of this document, the drawings address the right hip, right sacroiliac, right lumbo-sacral areas. Obviously, left sided configuration is the same with left sided orientation.

The following brace configurations are examples of straightforward anatomical placement. Many other possible configurations, including proprioceptive neuromuscular facilitation patterns can be made to suit individuals, albeit using the same brace components.

The length and width of the neoprene straps can vary depending upon each individual's needs. For this document the neoprene straps are narrow.

FIG. 1: starting position, still motion—neutral

FIG. 2: side view

FIG. 3: posterior view

FIG. 4: pattern for constructing brace, reversible right side and left side

FIG. 5: donning technique

FIG. 6: bilateral application

FIG. 7: neoprene straps-kit components

Drawing 8: alligator Velcro kit component

FIG. 9: double sided patches, circular placement indicators applied to brace pattern

FIG. 10: moderately adducted thigh

FIG. 11: anterior view—anti-dislocation

FIG. 12: posterior view—anti-dislocation

FIG. 13: posterior view internally rotated

FIG. 14: anterior view internally rotated

FIG. 15: side view—assist or resist abduction

FIG. 16: flexion assist or extensor resist

FIG. 17: extensor assist or flexion assist

FIG. 18: sacroiliac support

FIG. 19: S/l (sacroiliac and L/S (lumbo-sacral) strap position

FIG. 20: combined sacroiliac and lumbo-sacral strap position reach to anterior

FIG. 21: exemplary use of several components used simultaneously

FIG. 22: rotation of the entire brace

FIG. 23: examples of optional firm and/or firm and molded insertable parts

FIG. 24: added therapeutic components examples

### BRIEF DESCRIPTION OF DRAWINGS 1-22

### DRAWING 1: Front Facing View in Neutral Starting Position Free of Tension

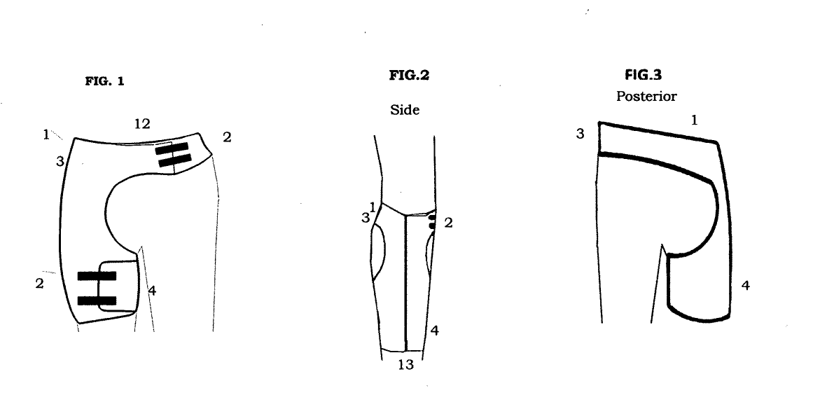

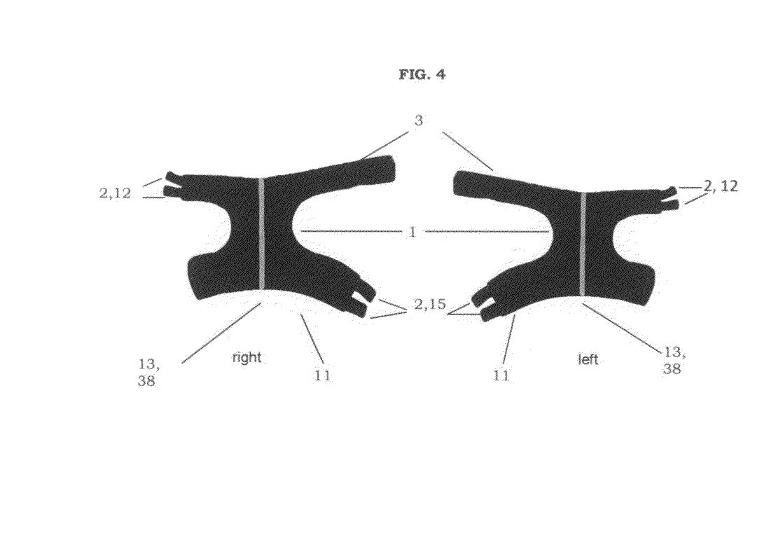

Drawing one depicts the embodiment of the hip brace in neutral position, configured for one (or 2-sided (FIG. 4)) neoprene body of the brace constructed in 2 or 4 parts and seamed together or cut from one piece and wraps around the lower torso and thigh. (FIG. 1,5) The body of the brace is constructed of elasticized neoprene or any elasticized fabric with tensile strength, which is secured with velcro closures having a hook side 17 to adhere to the neoprene or like adhering material or closure system 2. The placement and tension of velcro straps 2 and neoprene waist 3 and thigh bands 4 as well as the compression onto the entire area covered by the brace, or added firm components dictate the position of the thigh in reference to the pelvis and therefore the hip joint, sacroiliac, and lumbo-sacral spine.

DRAWING 2: Side View in Neutral Position without Tension

The side view of the right hip showing the position of the lateral seam aligned in midline. A pocket can be added to receive a stay. This is the beginning position when first donning the brace. (reference points 1,3,13,11,12,2)

### DRAWING 3: Posterior View

The posterior portion of the brace stretches from waistband 3 over the individual's sacrum and reaches distally to contain the sacro-iliac joint and courses down to the thigh 4.

The problematic hip is covered by the neoprene held under tension by the dynamic tensile nature of the elasticized neoprene fabric and the Velcro closures 15.

### DRAWING 4: Reversibility of Brace Construction Pattern

The reversible pattern of the neoprene body 1, (FIG. 1) of the brace is shown with the removable Velcro straps 2,17, on the ends of the waist and thigh bands 3,4,13,11,12 with both surfaces being receptive to the hook and loop of the Velcro 2,10,17 or like adhering material closure system. Thus, if double-sided unbroken loop neoprene is used, once flipped and Velcro or other adhering optional components removed and reversed, it fits the other hip. Or, it can be made singularly as right or left and lined with soft fabric, although unnecessary, as the UBL (unbroken loop material) surface is very soft.

For close customization for individual people, the neoprene or other elasticized fabric with tensile strength can be cut proportionally to follow the pattern (FIG. 4). The Velcro strap length can also be cut. 3,4

### DRAWING 5: Donning

Donning the hip brace body as in FIG. 1:

Utilizing the properties of elasticity, tensile strength, a neoprene or other like fabric and Velcro hook and loop construction or like closure system 17, the brace is put on 1,2 by wrapping and drawing the top waist 3,11 band 4,12 around the waist while simultaneously stretching it firmly in the direction shown while keeping the midline seam 13 traced upon the side. The distal end of the waist band with the Velcro hook end 2, is then pressed onto the body of the brace 17 at the waist or just below 12 or thigh band 4 is then drawn and wrapped firmly around the thigh 11 fixing the end onto the front of the thigh portion 5. Subsequently, the thigh 4 neoprene piece is then pulled down distally 14 where the thigh circumference narrows and re-tightened there.

### DRAWING 6: Bilateral

Two braces can be applied simultaneously to each side if both sides have problems, even if those problems are different. There will then be eight Velcro straps adhering in pairs of two 15 using a common waist band. (16,7,12,11)

### DRAWING 7: Kit Component 38

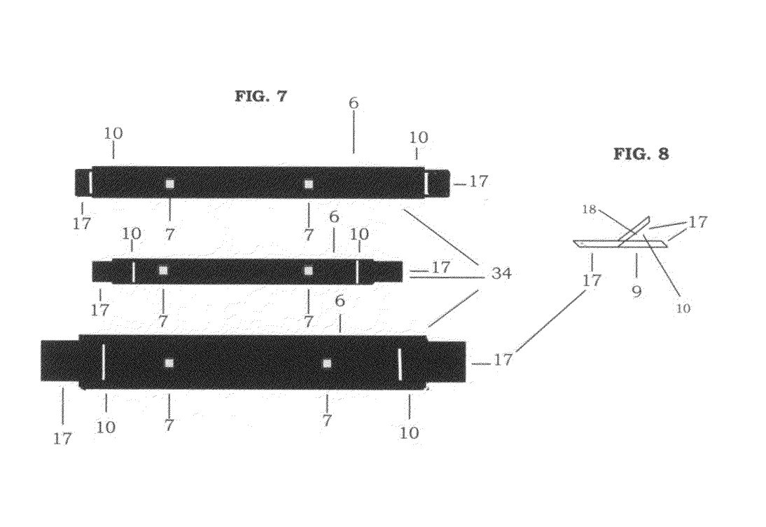

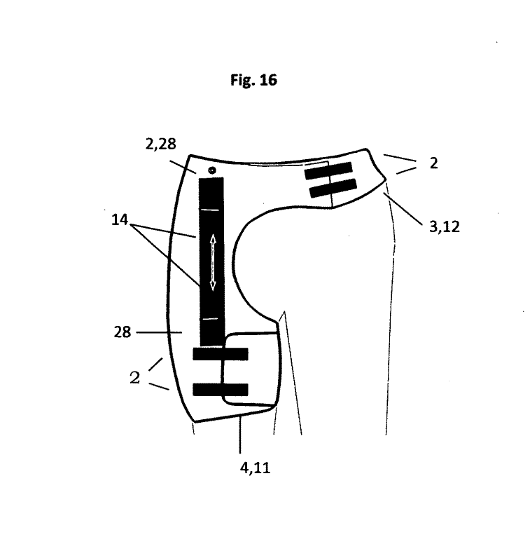

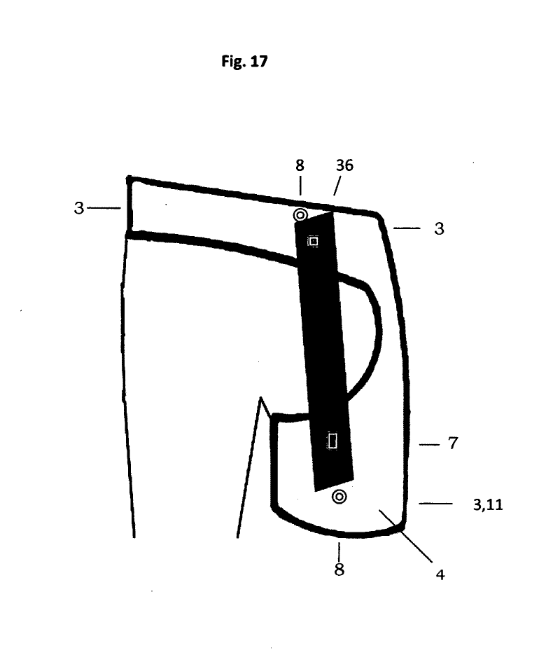

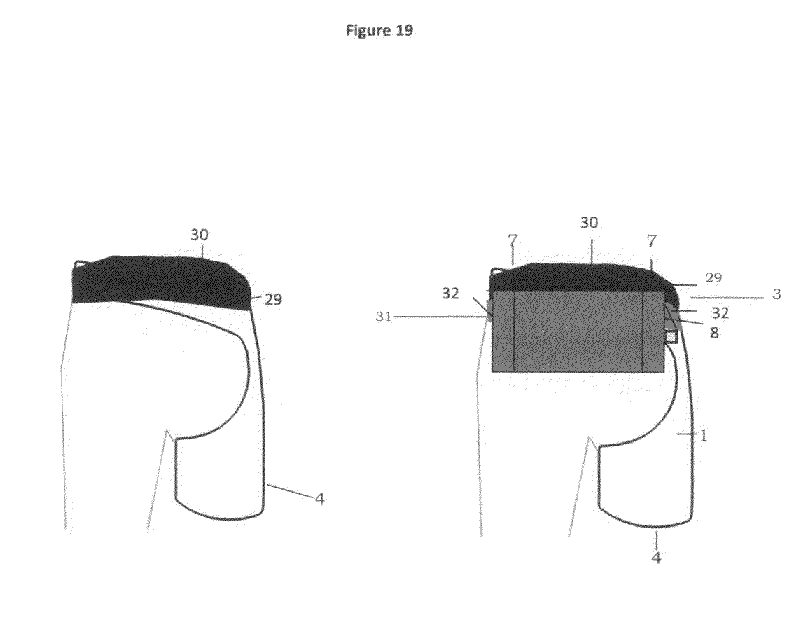

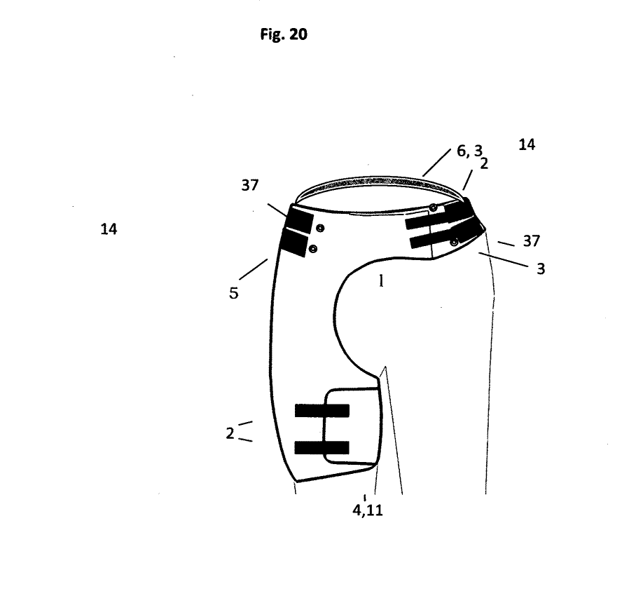

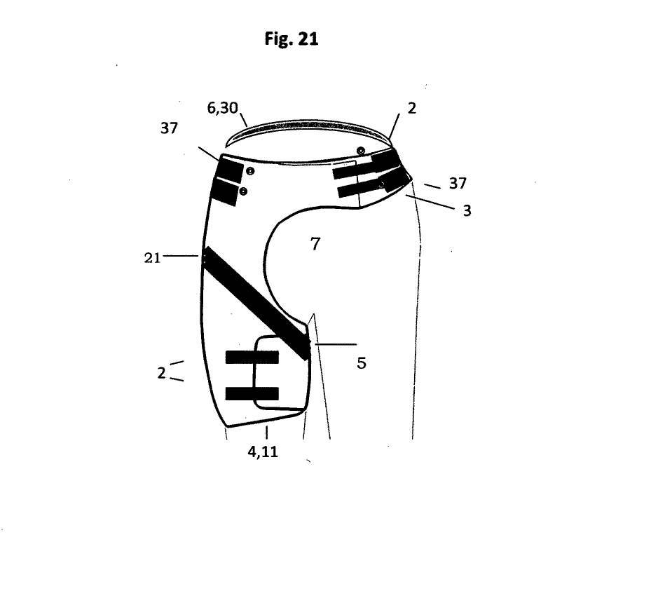

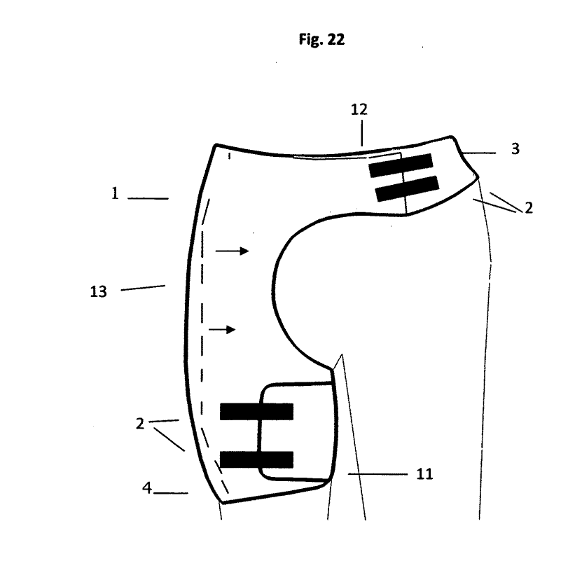

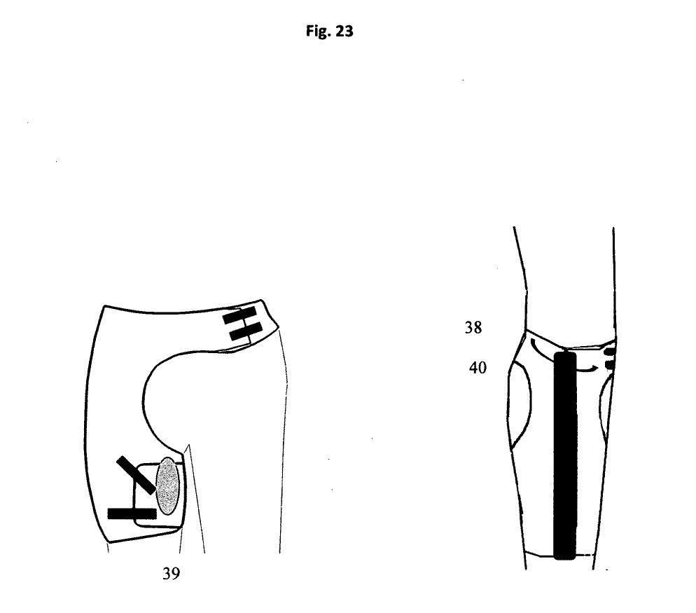

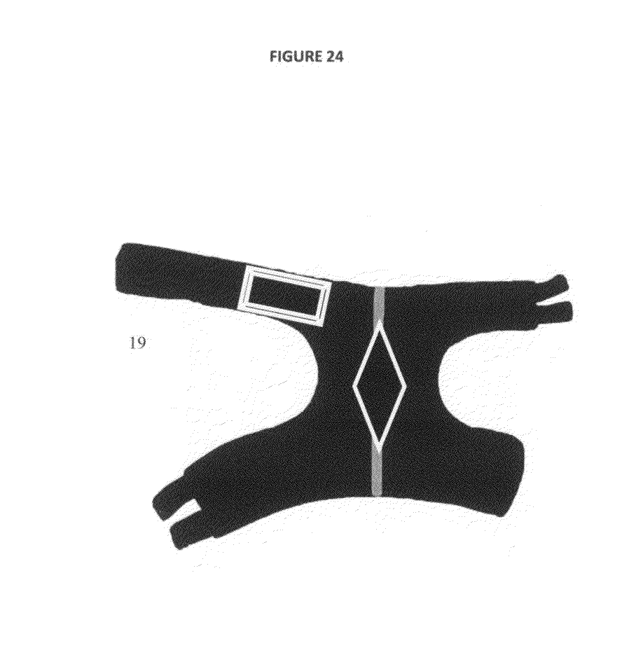

Depicts samples of the neoprene ancillary straps, 6 or like material with properties of elasticity and tensile strength with velcro hook and loop ended straps 17 (FIG. 7,8) or other closure system. The hip brace body hereto described is receptive to the adherence of Velcro 10,17 anywhere on the surface. These ancillary straps serve to fine tune positioning and supportive adjustments by being stretched and attached to various configurations that stabilize, assist, resist, lift, and secure desired position (FIG. 11-21). These ancillary straps can be used singly or in combination. All Velcro is totally removable 10 allowing for precise adjustment, tension and exercise capability. The amount of adjustment depends on placement and stretch 14 as well as coverage. They can be varying widths and lengths 34, stretched minimally to maximally.

### DRAWING 8: Alligator Type Velcro Closures; Kit Components[38]

To make the brace totally adjustable, the Velcro that attaches to the body of the brace can be double-ended Velcro alligator mouth 17,9,18. It wraps around ends of the waistband and the thigh band, as well as the ancillary straps 18, 10. The Velcro alligator type closures can be moved and re-attached for positional adjustment or switching it around for the other hip when using double-sided unbroken loop neoprene. 10

### DRAWING 9:—Kit Components

Double-sided patches of Velcro 7, 17 are used anywhere on doubled sided neoprene to hold straps or parts of the bands from slipping. When a patch is placed, the sticky hook sides adhere to the bottom of one component to the top of the other. No undesired movement of the straps occurs and therefore holds in place for the desired purpose and adjustment for the time wearing the brace 35. Circular Velcro patches 8 are placed strategically onto the body of the brace to avoid confusion and remind the individual where the optimum placement 10,11,12,13,15,16,17,19,20 of the ancillary neoprene with Velcro closure straps 2,5, or any Velcro placement can be marked with a dot. A fabric pouch 19 made of soft, breathable, material and adhered onto the neoprene brace with Velcro [38]. Inside the pouch can be hot, cold, or compression packs or pads, magnets, electrodes, compression pad, bandage and other optional therapies. A pouch can be worn outside as well to contain small belongings. (additional Reference points 1,3,4,14)

Additional firm and/or molded shapes can be inserted/glued/sewn into a neoprene configuration for added pressure in the desired direction.

### DRAWING 10: Moderately Adducted Thigh

For the hip that moderately internally rotates or adducts in flexion, the superior thigh Velcro 2,5, is applied at a 45 degree angle, gently drawing the thigh into abduction and external rotation 20. [additional Reference points 1,3,12,11,4,14] The resulting gait will be more toward neutral position.

### DRAWING 11: Anterior View—Anti-Dislocation

For the dislocating or subluxing hip, especially post surgically; bio-mechanically, the hip must be positioned in external rotation and abduction[6,21]. Starting in neutral position (FIG. 1) An ancillary neoprene strap 6 with Velcro closures 2, or is placed on the distal medial surface and drawn up and across the thigh to attach 5 on the waist band 4 on the opposite back side 14. Reference points 1,2,35,24,8,4,11 [,2,6,1]

### DRAWING 12: Posterior View—Anti-Dislocation

Viewing FIG. 11 from the posterior, the attachment of the ancillary strap with Velcro ends is indicated with the dot marker [22]. The strap is stretched and pulled 21 upward 25, 22 and attached on the waistband left of center, pull toward extension 23. The most prevalent provocative position for dislocation is prevented by this configuration. (Reference points 12,8,6,1,3,7,[4,11)

### DRAWING 13: Posterior View Internally Rotated

For the internally rotated hip, an ancillary Velcro ended strap, is first placed on the posterior medial lower thigh wrap 24; stretched pulling anteriorly 25 and toward the opposite anterior side. 26, 1,3,12,6,4,11,8

DRAWING 14: Anterior View Externally Rotated to be Pulled into Neutral or Slight Internal Rotation.

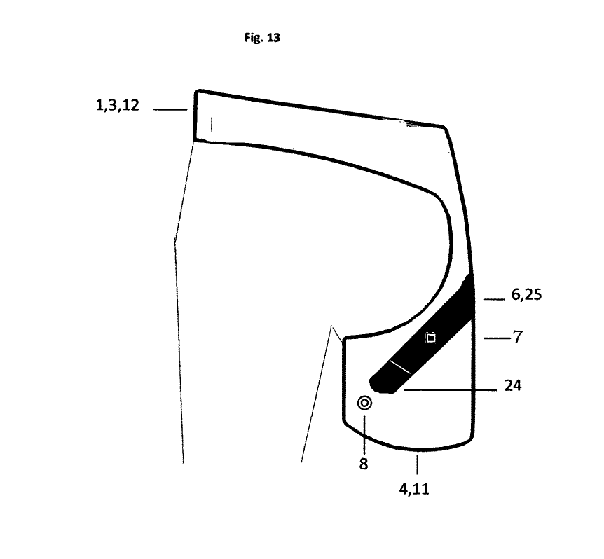

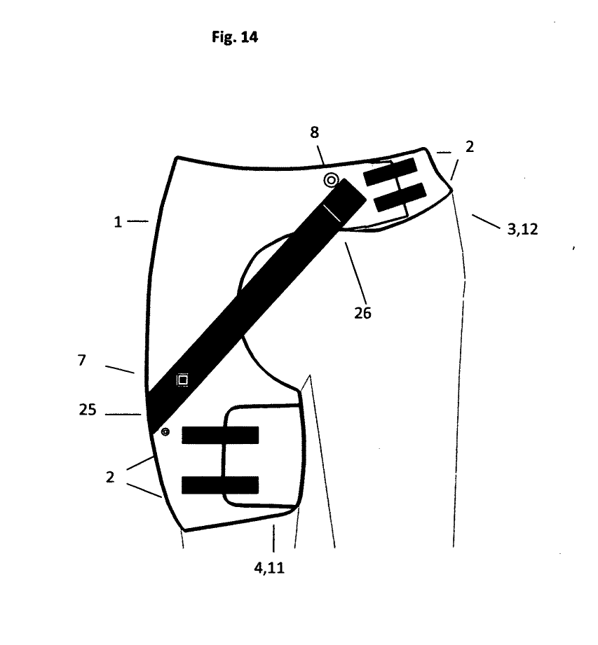

Continued stretch of the neoprene strap FIG. 13 is pulled anteriorly and upward to attach upon the waistband to the left of center 14 where marked by the dot marker 8,4,11,24,7,6,25 guiding the thigh into more internal rotation. Under the strap is a double-sided Velcro patch 7 to prevent movement of the strap. 1,3,2,12,26

### DRAWING 15: Side View—Assist or Resist Abduction

A neoprene strap with Velcro-ended strap (FIG. 7) is placed firmly on the waistband and stretched to the distal end of the lateral side. The tension produced by the pull of the fore mentioned strap assists the leg into abduction for those with weakness of hip abductors or, as an exercise resisting adduction, thereby strengthening the adductors. 27,14,11 (additional Reference points 1,3,12,4,6)

### DRAWING 16: Flexion Assist or Extensor Resist

An ancillary neoprene strap ((FIG. 7) or like material with Velcro 2 or like closure material is anchored on the anterior waistband 3, pulled 14 along the anterior of the waist band 12 and stretched 14 to just above the Velcro on the anterior thigh 4 and adhered there. The direction of pull acts to pull 14 the thigh 11 capitally 28.

### DRAWING 17: Extensor Assist or Flexion Assist

An ancillary neoprene (FIG. 7) or like strap is anchored with Velcro 2 onto the posterior waistband 3, 36 and stretched 14 caudally and adhered to the lower portion of the posterior thigh 4 band 11, marked by small patch 8 marker.

### DRAWING 18: Sacroiliac Strap

An ancillary neoprene strap with Velcro ends 6 or other like materials is stretched 14 across the S/l joints, reaching around to adhere onto the medial or medial frontal area on the waist 3 band 32. (additional Reference points 30,1,11,4,35)

### DRAWING 19: Lumbo-Sacral Strap Position, L/S Plus S/l Together

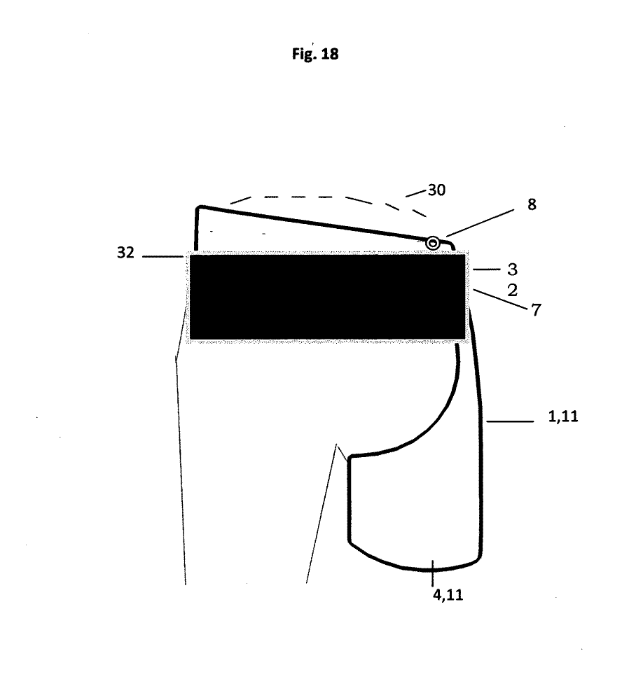

Each of two neoprene ancillary straps 6 with Velcro closures 5 or like materials applied one at a time; one anchored on the upper, L/S and the other on the lower waist band S/l 3 and drawn around the back to attach on the other side 32. The exerting pull and direction of tension 14 is to support the sacroiliac joints 31 and lumbo-sacral junction 33. (FIG. 18)

### DRAWING 20: Combined Sacroiliac and Lumbo-Sacral Strap Position Reaching Anterior Depiction of the Anterior and Lateral View of the Sacroiliac and Lumbo-Sacral Attachments 37.

Each of two neoprene ancillary straps 6 with Velcro closures 2, or applied one at a time; one anchored on the upper and the other on the lower waistband 3 and drawn around the back 12, 14, 33 to attach on the other side. The exerting pull and of tension is to support the sacroiliac joints and lumbo-sacral junction. ((FIG. 18)

DRAWING 21: an example of concomitant use of several components simultaneously: Reference points 37,6,30,2,3,4,11,21,35
DRAWING 22: rotation of the entire brace. To fine tune and customize positioning, the entire brace (FIG. 1) can be rotated left or right rotating therefore moving the lateral seam 14. (Reference points 1,13,2,4,11,2,3,12) Many other possible configurations, including proprioceptive neuromuscular facilitation patterns can be made to suit individuals, albeit using the same brace components.

### DRAWING 23:

Examples of insertable or fixed firm parts

### DRAWING 24:

Examples of some options to insert therapuetic treatment; ie; hot or cold packs, electrodes, etc

### LIST OF FIGURE REFERENCE NUMBERS

1 neoprene body of brace
2 Velcro closures for the singular brace demonstrating adjustment tab attaching in the front
3 waist band
4 thigh band
5 Velcro adhere to any part of brace
6 1 of 3 or more separate neoprene straps w alligator of a variety of lengths and widths
7 small Velcro double sided anti-slip patches
8 circular placement patches
9 alligator type closures line indicates side that removes for normal use
10 Velcro, alligator move and remove
11 thigh band wraps around the thigh
12 waist band wraps around the waist
13 midline seam for anatomical alignment reference
14 direction of pull to apply and set level of force exerted by strap
15 velcro straps with hooks adhered to brace in bilateral and combined l/s-s/l application
16 2 Velcro straps underneath
17 Velcro hook surface
18 alligator wrapping around end
19 pouch Velcroed onto neoprene to contain therapies
20 strap stretched firmly & adhered at 45 angle
21 strap wrapped to pull into ext. rot., abduction after anchored on thigh band
22 anchor spot for application of 21
23 attach end spot for anti-dislocation setup with extension
24 start spot for strap to pull into int. rot
25 continued wrap of strap
26 end spot for int. rot. on the front
27 stretched strap for abduction assist or add resist
28 strap for flex assist or ext resist adhered here
29 attachment spot for l/s wrap
30 arch of course of l/s strap
31 underlying waistband and/or s/l band
32 sacroiliac area under waistband and L/S support
33 double strap s/l, l/s—folded back
34 varying strap widths and lengths
35 dbl sided patches on the underside shown from beneath
36 beginning anchor of ext assist or flex. resist anchor
37 si/l/s anterior strap over lapping, adhering
38 stay/channel for stayss
39 insertible firm components—example vastus medialis pressure
40 Velcro strap that wraps to further secure the stay

### DETAILED DESCRIPTION OF THE INVENTION

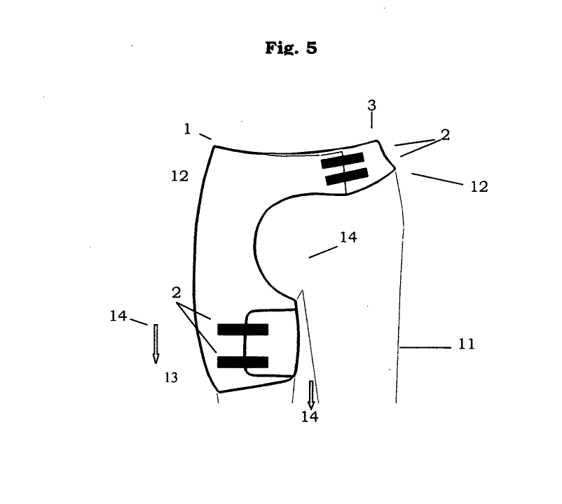

This invention is a hip brace constructed of soft materials (FIG. 1) 1,2,3,4,6,9, and optional hard components 19, 38,39, that provides motion control, joint support, joint protection, dislocation/subluxation prevention, pain management, proprioceptive cueing, pre and post-surgical support and protection; sacroiliac/lumbo-sacral support including radiculopathy; athletic support and protection of the hip joint and/or lumbo-sacral and/or sacral-elegiac joints on and off the field; means to contain therapeutic modalities and for exercise and pain management of all of these areas. Also, for athletes and active people in need of hip, sacroiliac and lumbo-sacral joint protection and support in the home, gym, ambulatory activity and athletics on and off the field. The brace body (FIG. 1) can be made constructed of single or double sided neoprene unbroken loop (UBL) or other material with the properties of elasticity and tensile strength receptive to Velcro 2 hook and loop 5,10 or other closure system (FIG. 5). With the use of double-sided unbroken loop neoprene, the right sided brace can be flipped to a left sided brace (FIG. 4) by removing the alligator Velcro closures 9,10 (FIG. 8) and re-attaching them back onto the other side. A single sided brace (FIG. 1, 5) can also be constructed of neoprene with Velcro 9, 10 or with sewn down straps. The single-sided neoprene comes with a bonded soft surface on the side that faces the skin. For close customization for individual people, the neoprene 1, can be cut proportionally to fit.

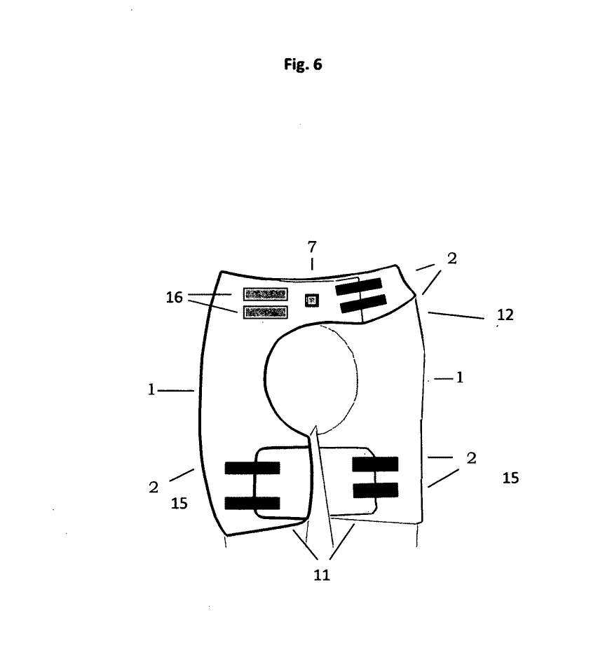

The basic embodiment of the brace (FIG. 1) provides adequate support for most conditions. However, the embodiment of the brace plus other components, known as the 38 such as additional straps 6, anti-slip patches 7, markers 8 that remind one where to place the components day to day, enclosures for a variety of functions with a stay, firm compression piece, hot or cold packs, electrodes, ointments for iontophoresis or normal ointment patch, etc. (FIG. 1-9,22,23)

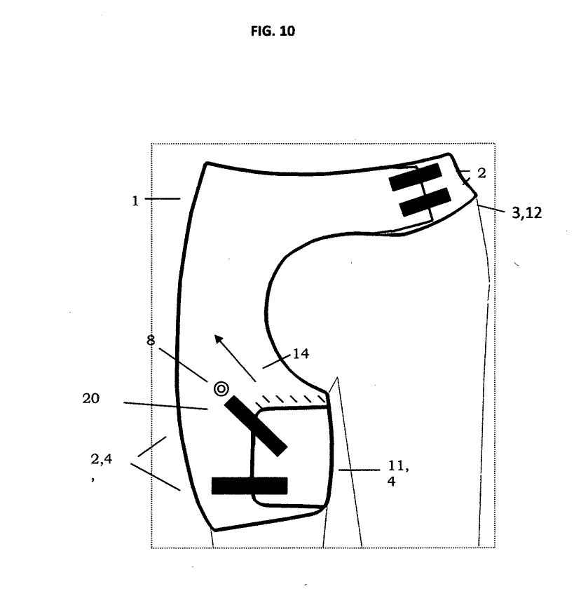

Dislocation and subluxation can be prevented by this brace (FIG. 10,11,12). Dislocation and subluxation continue to be the most common, difficult, and potentially frustrating postoperative complications after total hip arthroplasty. Various predisposing causes and associated factors have been suggested. These include malposition of the components, gluteal muscle deficiency, soft-tissue imbalance, and neuromuscular disorders, instability or imbalance of the sacroiliac and/or the lumbo-sacral joints are all addressed by this hip bracing device (FIG. 11,12,18,19, [19],20, 21). The hip joint, the sacroiliac and the lumbo-sacral joint contained within the brace are also secured by the properties of elasticity and fabric tensile strength of the neoprene 1, or elasticized fabric with like properties. The tension of the ancillary strap can be increased and decreased as per the muscular capability and specific chosen anchor spots 22, 14 Velcro hook and loop closures 2,5, or like material with its ability to endure pulling and stretching and still adhering 14 adds to the capability of this brace.

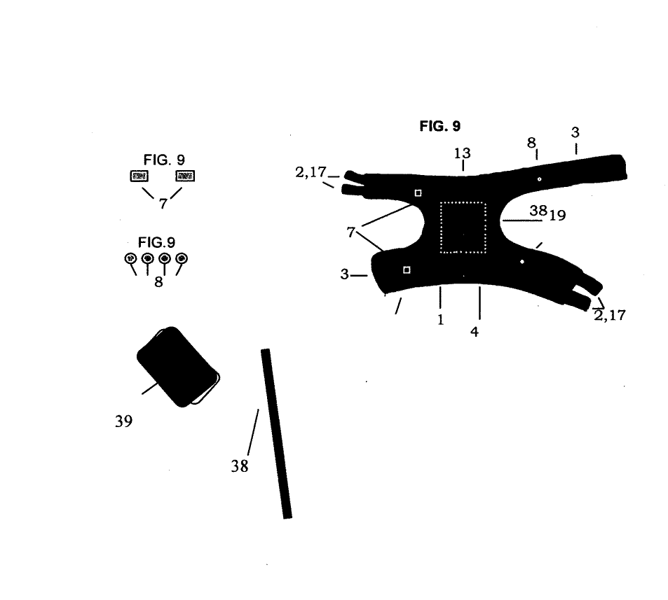

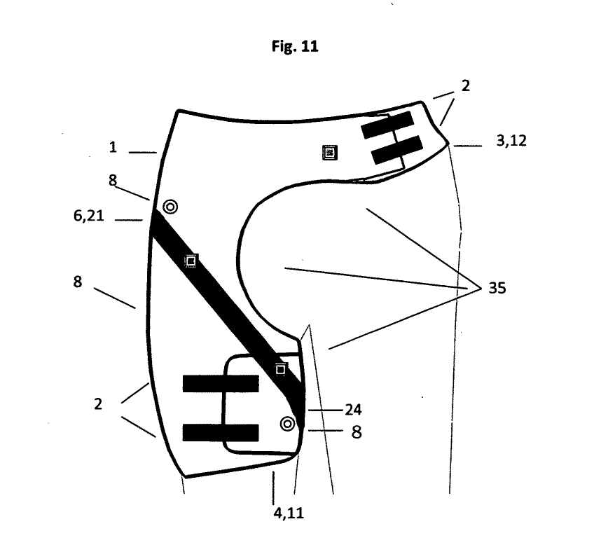

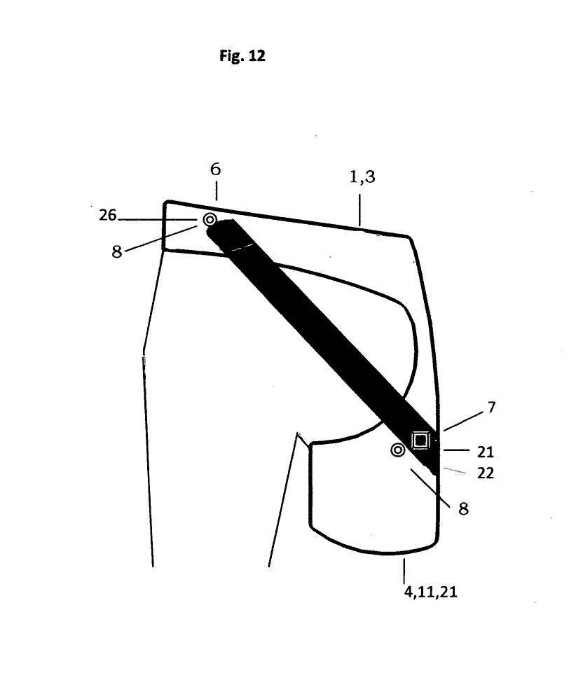

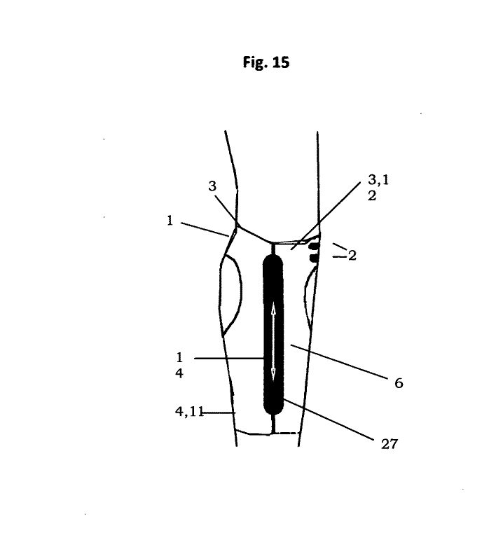

The Velcro holds fast 5,9 yet allows motion in all planes 2,5,10. In addition to and by virtue of separate neoprene, Velcro ended ancillary straps (FIG. 7) that adhere anywhere on the 5 body of the brace, the hip joint is well supported and positioned. There are multiple (FIG. 12,1,3,14,15,16,1,7,18,19,20,21,22,23) numbers of configuration possibilities which render advantageous support and control of movement for the individual and also by preventing undesirable aberrant motion. By securing motion within the brace configuration and targeting of attachment areas, there is improved alignment which the body can use to structure balanced strength training, and as such be used as ambulatory exercise and/or positioning for resistive and/or assistive exercise (FIG. 15,16,17). Individuals and especially athletes with vulnerable hip/sacroiliac/lumbo-sacral joints can wear this brace on and off of the field for support, protection, proprioceptive cueing, and for exercise. This brace is very comfortable and can be worn by males or females under or over clothing. Ancillary neoprene straps 6 with Velcro closures 2,5 at their ends 9,10 can be added and affixed to the neoprene or like materials, body of the brace. Each individual can be well supported by customized strategic placements of these added straps 21,22. The ancillary straps can adhere anywhere onto the neoprene or like material, brace 5. It can be configured to form a resistance and/or assistance to exercise system for the hip, sacroiliac and lumbo-sarcral joints or for general conditioning of these areas. Ancillary straps 6 can be linked together for the larger person or cut down for the smaller person by removing the Velcro 10 (FIG. 9) and shortening the neoprene. All of the Velcro/and ancillary neoprene straps are completely removable from both sides (FIG. 7). The brace can also contain a compression pack, ice or heat packs and/or magnets/electrodes within an inside pouch or placed on the outside (FIG. 9) or simply held by the elastic neoprene for application to the hip joint or sacroiliac/lumbo-sacral joints. The ancillary strap length can also be cut to fit the individual's body dimensions. The placement and tension 14 of these Velcro and/or neoprene straps on the body of the brace (FIG. 1) dictate the position of the thigh in reference to the pelvis and therefore the hip joint, sacroiliac, and lumbo-sacral spine. There are a variety of ways to place the ancillary Velcro straps (FIG. 7,8,9) for different applications. More than one strap can be used simultaneously. An additional property of this brace using elasticized fabrics is that it exerts pressure onto the hip and entire skin surface area covered by the materials. This pressure provides sensory stimulation onto the area. The brain/body registers this as awareness that the area is problematic and needs to be protected and oriented to where these body parts are in space, IE, proprioception, which cues the body to be vigilant to the structural vulnerability of unstable, injured, painful, poorly aligned joints or tissues of the lower quadrant, IE hip, sacroiliac, lumbo-sacral joints. Using neoprene straps to support the lumbo-sacral spine and sacroiliac simultaneously doubling with two over-lapping straps 31,37. The brace can also contain hot and cold packs, compression pads, electrodes in the pouch that affixes onto the interior on the midline next to the hip joint 19 or onto the exterior.

Men or women can wear this described brace and can be reversed by removing the Velcro (FIG. 7,8) and turning it around fit the other hip; left or right (FIG. 4). The design and the soft materials conform to the contour of an individual's body.

### LIST OF REFERENCE NUMBERS

1 neoprene body of brace
2 Velcro closures for the singular brace demonstrating adjustment tab attaching in the front
3 waist band
4 thigh band
5 Velcro adhere to any part of brace
6 1 of 3 or more separate neoprene straps w alligator of a variety of lengths and widths
7 small Velcro double sided anti-slip patches
8 circular placement patches
9 alligator type closures line indicates side that removes for normal use
10 Velcro, alligator move and remove
11 thigh band wraps around the thigh
12 waist band wraps around the waist
13 midline seam for anatomical alignment reference
14 direction of pull to apply and set level of force exerted by strap
15 velcro straps with hooks adhered to brace in bilateral and combined l/s-s/l application
16 2 Velcro straps underneath
17 Velcro hook surface
18 alligator wrapping around end
19 pouch Velcroed onto neoprene to contain therapies
20 strap stretched firmly & adhered at 45 angle
21 strap wrapped to pull into ext. rot., abduction after anchored on thigh band
22 anchor spot for application of 21
23 attach end spot for anti-dislocation setup with extension
24 start spot for strap to pull into int. rot
25 continued wrap of strap
26 end spot for int. rot. on the front
27 stretched strap for abduction assist or add resist
28 strap for flex assist or ext resist adhered here
29 attachment spot for l/s wrap
30 arch of course of l/s strap
31 underlying waistband and/or s/l band
32 sacroiliac area under waistband and L/S support
33 double strap s/l, l/s—folded back
34 varying strap widths and lengths
35 dbl sided patches on the underside shown from beneath
36 beginning anchor of ext assist or flex. resist anchor
37 si/l/s anterior strap over lapping, adhering
38 stay/channel for stayss
39 insertible firm components—example vastus medialis pressure
40 Velcro strap that wraps to further secure the stay

1. A dynamic hip brace to maximize a positive positioning and aligning of the thigh and torso, thereby controlling the relationship of the aspects of the hip joint, lumbo-sacral spine and sacroiliac, comprising:
a pelvic girdle having an upper portion configured for defining an upper opening for the wearer's waist, a lower portion configured for defining a lower opening for the wearer's hips, and a posterior portion between mid-lateral lines of the pelvic girdle and configured for engaging the wearer's lower back when the pelvic girdle is worn by the wearer; at least one thigh cuff defining lower and upper openings for the wearer's thigh;
means for generating an elastic tensile force between the one piece pelvic girdle and the thigh cuff in a longitudinal direction of the hip brace so as to hold the wearer's thigh to the wearer's pelvis;
wherein the elastic tension applied in the longitudinal direction by the dynamic hip stabilizer is between the pelvic girdle and the at least one thigh cuff so as to hold the wearer's thigh to the wearer's pelvis with sufficient tension to control excessive motion, provide hip stability by dynamically controlling and modifying hip motions through increasing tension as extremes of the hip motions are approached, and provide hip stability by constantly maintaining elevated abductor tension using ancillary neoprene straps and velcro; a means of inserting a solid stay to additionally enhance abductor/adductor control.
an unbroken loop (UBL) single-sided or double-sided neoprene body of the brace that wraps around the lower torso and thigh and attached via Velcro closures positioned to offer straight or angular placement;
a singular neoprene body with contiguous bands with an upper torso portion of the embodiment of unbroken loop neoprene that wraps around the waist and closes with one or a plurality of Velcro closures pressed firmly against the unbroken loop neoprene on the front side of the brace and a lower portion of unbroken loop neoprene that wraps around the thigh, then firmly and closes with one or a plurality of Velcro closures on the front side of the thigh;
unbroken loop ancillary neoprene straps ended with alligator or sewn on Velcro closures which adhere to the hip brace then stretched to the thigh in a variety of possible configurations and can be cut to size;
the alligator configured Velcro closures option which provide secure location by wrapping around the ends of neoprene as they adhere to the top and bottom of the neoprene strap or body of the brace and are removable for reversibility and custom sizing with double-sided neoprene;
optionally, double sided velcro patches that adhere to secure neoprene unbroken loop straps from sliding by sticking above and below the strap to the unbroken loop neoprene embodiment or for use between ancillary neoprene straps to maintain the position of added straps or even adhered to the waist and thigh bands when they overlap;
optionally, circular Velcro dot markers that are used to mark the target site where the unbroken loop neoprene straps attach to surface of the neoprene brace with Velcro;
optionally, a pouch that adheres to the underside of the hip brace to hold an assortment of therapies such as a hot pack, cold pack, opening for a stay, electrodes, magnets, pressure bandage or alternatively can be worn on the outside of the hip brace;
means to apply compression onto the entire area covered by the brace to dictate the position of the thigh in reference to the pelvis and therefore the hip joint, sacroiliac, and lumbo-sacral spine when the posterior portion of the brace stretches from the Lumbar spine over the individual's sacrum and reaches distally to contain the sacro-iliac joint and courses down the thigh of the affected hip comprising;
an unbroken loop double-sided or, optionally single-sided, elasticized neoprene body of the brace patterned with two bands of unbroken loop elasticized double sided or, optionally single-sided, neoprene straps;

2. The dynamic hip brace of claim 1 wherein the waist and thigh bands are specifically configured to close in the front where easy adjustment for the wearer to don the hip brace independently wherein the hip brace prevents hip dislocation, provides support to the hip, lumbo-sacral spine, and sacroiliac joints by using the placement and tension of four Velcro straps, neoprene waist and thigh bands, ancillary neoprene straps, closures from the waist band, closures from the thigh band providing compression onto the entire area covered by the brace to dictate the position of the thigh in reference to the pelvis and therefore the hip joint, sacroiliac, and lumbo-sacral spine when the posterior portion of the brace stretches from the Lumbar spine over the individual's sacrum and reaches distally to contain the sacro-iliac joint and courses down the thigh of the affected hip.

3. The dynamic hip brace of claim 1 can have an alternate construction using single sided neoprene whereas the loop surface is on the exterior only with a bonded soft fabric lining.

4. The dynamic hip brace of claim 1 optionally provides the reversible pattern of double-sided unbroken loop neoprene body of the brace with the removable alligator Velcro straps, on the ends of the waist and thigh bands with both surfaces being receptive to the hook of the Velcro and ending on the front of the brace.

5. The dynamic hip brace of claim 1 when made from double sided unbroken loop neoprene can be flipped for either left or right hip application.

6. The dynamic hip brace of claim 2 made of neoprene material, single or double-sided, and cut to a specific configuration as in the pattern wherein the waist and thigh bands are specifically configured to close in the front providing easy adjustment for the wearer to don the hip brace independently without help.

7. The dynamic hip brace of claim 2 contains unbroken loop neoprene straps with Velcro closures of any desired length or width that wrap in the prescribed direction and allow for access on the front side of the brace.

8. The dynamic hip brace of claim 2 wraps and overlaps around the waist and wraps and overlaps around the thigh.

9. The dynamic hip brace of claim 2 wherein the Velcro attachment closures onto the front of the waist and thigh of the embodiment, being secured by pressing the Velcro hook onto the unbroken loop fabric.

10. The dynamic hip brace of claim 1 contains an assortment of ancillary unbroken loop straps with Velcro closures that can be applied to the embodiment to allow for normal gait and for exercise in a secure position and alignment.

11. The dynamic hip brace of claim 2 wherein the ancillary straps use the length of these ancillary straps used singly or in combination to determine the amount of adjustment and efficacy and final end placement by formulating the needed amount of stretch from neutral then stretched in a chosen configuration as dictated by the problematic area and the desired correction by using the elasticity of the neoprene of the embodiment and the elasticity of the straps to pull or secure the hip, lumbo-sacral spine, sacroiliac joints into the desired position.

12. The dynamic hip brace of claim 11 wherein the elasticized materials are adjusted when the brace is put on and the bands are tightened, the thigh band is then pulled downward and is then re-tightened where the thigh circumference is smaller, and when then released, the exerting upward force seats the femoral head into the pelvic acetabulum by taking advantage of the elasticized neoprene and Velcro with the tensile force of the bracing material providing freedom of movement.

13. The dynamic hip brace of claim 11 provides positioning into external rotation positioning with use of Velcro only pulling strategically into external rotation, abduction with the brace body held in neutral but the upper thigh Velcro hook strap pulled firmly upward attaching to the neoprene loops at a 45 degree angle leading the femur into external rotation when the leg is moving forward in a normal gait pattern, the hip conveniently using the momentum from the leg swing phase.

14. The dynamic hip brace of claim 11 wherein an ancillary neoprene strap with Velcro closures is placed on the distal medial surface of the same thigh and drawn up and across around to the backside the thigh to attach on the posterior waist band and the attachment target of the ancillary strap with Velcro is stretched and pulled upward and attached on the posterior waistband on the opposite side to prevent the provocative position for dislocation.

15. The dynamic hip brace of claim 11 wherein an ancillary neoprene/Velcro ended strap to the starting point of posterior/medial thigh and pulling the strap tension forward anteriorly and upward to attach upon the waist band to the left of center guiding the thigh into adduction and internal rotation.

16. The dynamic hip brace of claim 11 wherein the neoprene Velcro ended straps can be applied to the body of the brace or even to other neoprene ended straps to exert the desired pull and position as combinations of applied positioning and strap locations, direction of pull, and strength of pull are infinite but discernible by the individual with hip pain, physical therapist, and physician, and customized for each individual.

17. The dynamic hip brace of claim 13 wherein localized areas or spots of corrosion of the femoral head which is far less painful when weight bearing pressure is off when ambulating with this brace and with adjustment of the brace and components whereas the thigh/leg relationship can be held so as to avoid pressure on the tender spots thus relieving arthritic pain.

18. The dynamic hip brace of claim 11 wherein using straps for the lumbo-sacral spine and hip combines to elevate the leg enough to lift the leg, taking pressure off of the nerves descending into the leg causing radicular pain.
